(19) United States
(12) Patent Application Publication
(10) Pub. No.: US 2025/0057562 A1
(43) Pub. Date: Feb. 20, 2025

(54) PERICARDIAL TRANSECTION DEVICE WITH RETRACTABLE CUTTING APPARATUS

(71) Applicant: EDWARDS LIFESCIENCES CORPORATION, Irvine, CA (US)

(72) Inventors: Daniel James Murray, Orange, CA (US); Jose Islas, Corona, CA (US); Arvin T. Chang, Yorba Linda, CA (US)

(21) Appl. No.: 18/940,693

(22) Filed: Nov. 7, 2024

Related U.S. Application Data
(63) Continuation of application No. PCT/US2023/022947, filed on May 19, 2023.
(60) Provisional application No. 63/346,317, filed on May 26, 2022.

(51) Int. Cl. A61B 17/32; A61B 17/00; A61B 17/3209; A61B 17/3211; A61B 18/00; A61B 18/14

(52) U.S. Cl. CPC A61B 17/320016; A61B 17/3209; A61B 18/1492; A61B 2017/00243; A61B 2017/32113; A61B 2018/00601; A61B 2018/1412

(57) ABSTRACT

A medical device for creating elongated incisions within a pericardium is provided. The device also includes an incision device operably coupled to a distal end of a catheter. The device also includes a retractable cutting apparatus disposed within an incision channel of the incision device. The retractable cutting apparatus includes a gear-actuated blade structured to move between a retracted and a deployed position. In an instance in which the gear-actuated blade is in the retracted position, the gear-actuated blade is disposed within the incision channel. In an instance in which the gear-actuated blade is in the deployed position, the gear-actuated blade at least partially protrudes from the incision channel. The retractable cutting apparatus also includes a torsion gear mechanism attached to the gear-actuated blade. The retractable cutting apparatus also includes a rotatable gear actuator that is structured to move the torsion gear mechanism.

### RELATED APPLICATIONS

This application is a continuation application of International Patent Application No. PCT/US2023/022947, filed May 19, 2023, which claims the benefit of U.S. Provisional Application No. 63/346,317, filed on May 26, 2022. Each of the foregoing patent applications are hereby incorporated by reference in their entireties.

### TECHNICAL FIELD

This disclosure is directed to devices and methods for cutting in the pericardium. The devices and methods are generally applicable to the treatment of heart failure, for example, heart failure with preserved ejection fraction (HFpEF) or reduced injection fraction (HFrEF) by introducing one or more incision lengths in a pericardium, e.g., a parietal layer.

### BACKGROUND

Pericardial restraint is a normal physiologic process that becomes exaggerated, for example, in some patients with heart failure with preserved ejection fraction (HFpEF) and causes the right heart to run out of space when filling, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

In an example embodiment, a medical device for creating elongated incisions within a pericardium is provided. The device includes a multi-lumen catheter including at least one lumen, a longitudinal axis, a proximal end, and a distal end. The device also includes an incision device operably coupled to the distal end of the multi-lumen catheter. The incision device includes an incision channel defined along the incision device. The device further includes a retractable cutting apparatus disposed within the incision channel. The retractable cutting apparatus includes a gear-actuated blade structured to move between a retracted position and a deployed position within the incision channel. In an instance in which the gear-actuated blade is in the retracted position, the gear-actuated blade is disposed within the incision channel. In an instance in which the gear-actuated blade is in the deployed position, the gear-actuated blade at least partially protrudes from the incision channel. The retractable cutting apparatus also includes a torsion gear mechanism attached to the gear-actuated blade. The torsion gear is structured to move the gear-actuated blade between the retracted position and the deployed position. The retractable cutting apparatus also includes a rotatable gear actuator that is structured to move the torsion gear mechanism.

In some example embodiments, alone or in combination with any of the previous example embodiments, the blade of the cutting apparatus is structured to be moveable between the retracted position and the deployed position upon placement of the incision device within a pericardium of the human body. In some example embodiments, alone or in combination with any of the previous example embodiments, the retractable cutting apparatus includes a force transferring gear mechanism. In some example embodiments, alone or in combination with any of the previous example embodiments, the retractable cutting apparatus includes a force transferring gear mechanism is a worm gear.

In some example embodiments, alone or in combination with any of the previous example embodiments, the retractable cutting apparatus includes an intermediate actuation member, and the intermediate actuation member is disposed between the force transferring gear mechanism and the torsion gear mechanism. In some example embodiments, alone or in combination with any of the previous example embodiments, torsion gear mechanism is a rack and pinion gear. In some example embodiments, alone or in combination with any of the previous example embodiments, a rack portion of the rack and pinion gear is attached to the gear-actuated blade. In some example embodiments, alone or in combination with any of the previous example embodiments, the blade is structured to move along a latitudinal axis upon actuation of the torsion gear mechanism.

In some example embodiments, alone or in combination with any of the previous example embodiments, a pinion portion of the rack is a pinion gear is attached to the rotatable gear actuator, and the rotation of the rotatable gear actuator causes the rack portion to move along the pinion portion of the rack and pinion gear. In some example embodiments, alone or in combination with any of the previous example embodiments, a pinion portion of the rack and pinion gear is attached to the intermediate actuation member.

In some example embodiments, alone or in combination with any of the previous example embodiments, the intermediate actuation member defines a proximal end and a distal end, and at least a portion of the force transferring gear mechanism is attached to the proximal end of the intermediate actuation member and at least a portion of the torsion gear mechanism is attached to the distal end of the intermediate actuation member.

In some example embodiments, alone or in combination with any of the previous example embodiments, one or more stabilizing members are provided along the multi-lumen catheter, and the one or more stabilizing members are structured to maintain the incision device in place during an incision by the gear-actuated blade. In some example embodiments, alone or in combination with any of the previous example embodiments, one or more stabilizing members are at least one stabilizing balloon. In some example embodiments, alone or in combination with any of the previous example embodiments, the one or more stabilizing member include a first stabilizing balloon and a second stabilizing balloon. In some example embodiments, alone or in combination with any of the previous example embodiments, the first stabilizing balloon, the second stabilizing balloon, and the blade are positioned approximately 120 degrees from one another around the catheter.

In some example embodiments, alone or in combination with any of the previous example embodiments, the one or more stabilizing member is one or more inflatable structures. In some example embodiments, alone or in combination with any of the previous example embodiments, the at least one stabilizing balloon is positioned to contact an epicardium upon positioning of the medical device within a pericardial cavity. In some example embodiments, alone or in combination with any of the previous example embodiments, the at least one stabilizing balloon is made out of a polymer material.

In some example embodiments, alone or in combination with any of the previous example embodiments, the stabilizing balloon is filled with saline. In some example embodiments, alone or in combination with any of the previous example embodiments, the at least one stabilizing balloon is filled with saline via a syringe. In some example embodiments, alone or in combination with any of the previous example embodiments, the at least one stabilizing member is a wire, loop, or shape-memory metal.

In some example embodiments, alone or in combination with any of the previous example embodiments, the gear-actuated blade is longer in a longitudinal direction that the stabilizing balloon in the longitudinal direction. In some example embodiments, alone or in combination with any of the previous example embodiments, the at least one stabilizing member and the gear-actuated blade are independently adjusted. In some example embodiments, alone or in combination with any of the previous example embodiments, the at least one stabilizing member and the gear-actuated blade are structured to be adjusted within the human based on the size of the pericardial cavity of the human.

In some example embodiments, alone or in combination with any of the previous example embodiments, the rotatable gear actuator is a torsion shaft. In some example embodiments, alone or in combination with any of the previous example embodiments, the torsion shaft of the rotatable gear actuator defines a proximal end and a distal end. In such an embodiment, at least a portion of the torsion gear mechanism is attached to the distal end of the torsion shaft. In some example embodiments, alone or in combination with any of the previous example embodiments, a torsion knob is disposed at the proximal end of the torsion shaft, and the torsion knob is structured to be manipulated to cause a torsional force on the torsion shaft.

In some example embodiments, alone or in combination with any of the previous example embodiments, the device also includes a multi-lumen catheter controller structured to move the rotatable gear actuator. In some example embodiments, alone or in combination with any of the previous example embodiments, the multi-lumen catheter controller is further structured to move the multi-lumen catheter. In some example embodiments, alone or in combination with any of the previous example embodiments, the multi-lumen catheter controller defines a handle.

In some example embodiments, alone or in combination with any of the previous example embodiments, the torsion knob is positioned on the handle of the multi-lumen catheter controller, and the torsion knob is structured to rotate relative to the handle of the multi-lumen catheter controller. In some example embodiments, alone or in combination with any of the previous example embodiments, the torsion shaft is structured to be manipulated by a handle controller.

In some example embodiments, alone or in combination with any of the previous example embodiments, upon movement of the blade into the deployed position, the retractable cutting apparatus is structured to be moved along the incision channel while the incision device remains stationary within the human. In some example embodiments, alone or in combination with any of the previous example embodiments, wherein the at least one stabilizing member is moved to a stabilizing retracted position after the gear-actuated blade is moved to the deployed position. In some example embodiments, alone or in combination with any of the previous example embodiments, the at least one stabilizing member is in the stabilizing retracted position in an instance in which the multi-lumen catheter is moved along the pericardial cavity.

In some example embodiments, alone or in combination with any of the previous example embodiments, the device also includes a sheath structured to moveably cover the incision channel. In some example embodiments, alone or in combination with any of the previous example embodiments, the sheath is structured to cover the incision channel in an instance in which the multi-lumen catheter is not positioned within human body. In some example embodiments, alone or in combination with any of the previous example embodiments, the sheath is structured to removably cover the one or more stabilizing members. In some example embodiments, alone or in combination with any of the previous example embodiments, the sheath is structured to removably cover the blade. In some example embodiments, alone or in combination with any of the previous example embodiments, the sheath is structured to removably cover the one or more stabilizing members and the cutting surface.

In some example embodiments, alone or in combination with any of the previous example embodiments, the multi-lumen catheter controller is structured to move the sheath between a covered position and an uncovered position, and the sheath covers the incision channel in the covered position. In some example embodiments, alone or in combination with any of the previous example embodiments, the sheath has a distal end being slidably locatable on the multi-lumen catheter. In some example embodiments, alone or in combination with any of the previous example embodiments, the sheath also includes at least one opening adjacent the distal end of the sheath. In some example embodiments, alone or in combination with any of the previous example embodiments, the distal end of the sheath is traversable along the multi-lumen catheter.

In some example embodiments, alone or in combination with any of the previous example embodiments, the at least one opening of the sheath is traversable along the multi-lumen catheter to cover the incision channel or to uncover the incision channel. In some example embodiments, alone or in combination with any of the previous example embodiments, at least a portion of the distal end of the sheath and at least a portion of the incision channel are radiopaque to align the distal end of the sheath and the incision channel.

In some example embodiments, alone or in combination with any of the previous example embodiments, the at least one opening of the sheath is traversable to align with the incision channel, allowing the blade to laterally project from the incision channel. In some example embodiments, alone or in combination with any of the previous example embodiments, the at least one opening of the sheath is traversable to align with the incision channel, allowing the actuator to cause the cutting surface to laterally project from the incision channel. In some example embodiments, alone or in combination with any of the previous example embodiments, the distal end of the sheath concurrently or sequentially allows the blade and the one or more stabilizing members to laterally project from the multi-lumen catheter.

In some example embodiments, alone or in combination with any of the previous example embodiments, the gear-actuated blade includes a cutting surface. In some example embodiments, alone or in combination with any of the previous example embodiments, the cutting surface is positioned to cut tissue upon being moved within the human body in an instance in which the blade is in the deployed position. In some example embodiments, alone or in combination with any of the previous example embodiments, at least a portion of cutting surface is reversibly adjustable laterally relative to the longitudinal axis of the multi-lumen catheter between a range of angles.

In some example embodiments, alone or in combination with any of the previous example embodiments, the cutting surface is parallel to the longitudinal axis of the multi-lumen catheter in retracted position and the deployed position. In some example embodiments, alone or in combination with any of the previous example embodiments, at least a portion of the retractable cutting apparatus is radiopaque. In some example embodiments, alone or in combination with any of the previous example embodiments, the blade is structured to have a sufficient force to puncture pericardial tissue in an instance in which the blade is moved from the retracted position to the deployed position.

In some example embodiments, alone or in combination with any of the previous example embodiments, the medical device is sterilized. In some example embodiments, alone or in combination with any of the previous example embodiments, the medical device can be sterilized via gamma sterilization. In some example embodiments, alone or in combination with any of the previous example embodiments, the medical device can be sterilized via ethylene oxide sterilization. In some example embodiments, alone or in combination with any of the previous example embodiments, the medical device can be sterilized via autoclave sterilization. In some example embodiments, alone or in combination with any of the previous example embodiments, the medical device can be sterilized via E-beam sterilization.

In some example embodiments, alone or in combination with any of the previous example embodiments, at least a portion of the blade is radiopaque. In some example embodiments, alone or in combination with any of the previous example embodiments, at least a portion of the one or more stabilizing member is radiopaque. In some example embodiments, alone or in combination with any of the previous example embodiments, at least a portion of at the cutting apparatus and the one or more stabilizing member are radiopaque.

In some example embodiments, alone or in combination with any of the previous example embodiments, the blade is structured to have a sufficient force to puncture pericardial tissue in an instance in which the blade is moved from the retracted position to the deployed position. In some example embodiments, alone or in combination with any of the previous example embodiments, the blade is assisted in cutting into the pericardium by a force provided by the one or more stabilizing member. In some example embodiments, alone or in combination with any of the previous example embodiments, the incision is linear. In some example embodiments, alone or in combination with any of the previous example embodiments, the incision is non-linear.

In some example embodiments, alone or in combination with any of the previous example embodiments, the multi-lumen catheter is steerable. In some example embodiments, alone or in combination with any of the previous example embodiments, at least a portion of the multi-lumen catheter is radiopaque. In some example embodiments, alone or in combination with any of the previous example embodiments, the device also includes an introducer positioned near the distal end of the multi-lumen catheter In some example embodiments, alone or in combination with any of the previous example embodiments, at least a portion of the introducer is radiopaque.

In some example embodiments, alone or in combination with any of the previous example embodiments, the at least one lumen includes a guidewire lumen. In some example embodiments, alone or in combination with any of the previous example embodiments, the device also includes a guidewire slidably positioned within the guidewire lumen.

In some example embodiments, alone or in combination with any of the previous example embodiments, the device also includes at least one nerve detection device. In some example embodiments, alone or in combination with any of the previous example embodiments, the at least one nerve detection device is located on the multi-lumen catheter. In some example embodiments, alone or in combination with any of the previous example embodiments, the at least one nerve detection device is located adjacent the incision device. In some example embodiments, alone or in combination with any of the previous example embodiments, the at least one nerve detection device is located on the puncturing tip. In some example embodiments, alone or in combination with any of the previous example embodiments, the at least one nerve detection device is located on the cutting surface.

In some example embodiments, alone or in combination with any of the previous example embodiments, the device also includes at least one nerve stimulation device. In some example embodiments, alone or in combination with any of the previous example embodiments, the at least one nerve stimulation device is located on the multi-lumen catheter. In some example embodiments, alone or in combination with any of the previous example embodiments, the at least one nerve stimulation device is located adjacent the incision device. In some example embodiments, alone or in combination with any of the previous example embodiments, the at least one nerve stimulation device is located on the puncturing tip. In some example embodiments, alone or in combination with any of the previous example embodiments, the at least one nerve stimulation device is located on the cutting surface.

In some example embodiments, alone or in combination with any of the previous example embodiments, the multi-lumen catheter includes a camera lumen. In some example embodiments, alone or in combination with any of the previous example embodiments, the device includes a camera structured to be positioned in the camera lumen.

In some example embodiments, alone or in combination with any of the previous example embodiments, at least a portion of the incision device includes an electrode. In some example embodiments, alone or in combination with any of the previous example embodiments, at least a portion of the cutting surface includes an electrode. In some example embodiments, alone or in combination with any of the previous example embodiments, the electrode is a wire. In some example embodiments, alone or in combination with any of the previous example embodiments, the wire is shaped as one or more arcs projecting laterally through the multi-lumen catheter along the longitudinal axis.

In some example embodiments, alone or in combination with any of the previous example embodiments, the cutting surface includes a scalpel. I In some example embodiments, alone or in combination with any of the previous example embodiments, at least a portion of the scalpel includes an electrode. In some example embodiments, alone or in combination with any of the previous example embodiments, the at least one electrode is electrically couplable to a source of radiofrequency energy or electrical current sufficient to cut, separate, scissor, or evaporate a portion of the parietal layer.

In some example embodiments, alone or in combination with any of the previous example embodiments, the device also includes a second electrode adjacent to the incision device. In some example embodiments, alone or in combination with any of the previous example embodiments, the second electrode is operably coupled to a source of radiofrequency energy or electrical current.

In some example embodiments, alone or in combination with any of the previous example embodiments, the cutting surface when laterally projected, faces away from the distal end and towards the proximal end of the multi-lumen catheter. In some example embodiments, alone or in combination with any of the previous example embodiments, at least a portion of cutting surface is reversibly adjustable laterally relative to the longitudinal axis of the multi-lumen catheter between a range of angles. In some example embodiments, alone or in combination with any of the previous example embodiments, the range of angles are reversibly adjustable for providing a scissor cut of parietal layer tissue.

In some example embodiments, alone or in combination with any of the previous example embodiments, a kit includes the medical device of various embodiments, a sheath, a guidewire, and a puncturing tip.

In another example embodiment, a method of creating elongated incisions within a pericardium is provided. The method includes placing a multi-lumen catheter within a human. The multi-lumen catheter includes at least one lumen, a longitudinal axis, a proximal end, and a distal end. The method also includes positioning an incision device within the pericardium. The incision device is operably coupled to the distal end of the multi-lumen catheter and the incision device includes an incision channel defined along the incision device. The method further includes deploying a blade of a retractable cutting apparatus disposed within the incision channel from a retracted position to a deployed position. In an instance in which the blade is in the retracted position, the blade is disposed within the incision channel. In an instance in which the blade is in the deployed position, the blade at least partially protrudes from the incision channel. The method still further includes moving the incision device remains stationary within the pericardium, and the blade causes an incision in the pericardium.

In some example embodiments, alone or in combination with any of the previous example embodiments, the creation of the at least one incision length through the pericardium causes a reduction in pressure exerted on a heart by the pericardium. In some example embodiments, alone or in combination with any of the previous example embodiments, the incision length is created in at least one of a parietal layer or fibrous layer of the pericardium. In some example embodiments, alone or in combination with any of the previous example embodiments, the incision length is created in adipose tissue or fat deposits. In some example embodiments, alone or in combination with any of the previous example embodiments, the at least one incision length is along a length or circumference of only the parietal layer of the pericardium. In some example embodiments, alone or in combination with any of the previous example embodiments, the at least one incision length is made in the parietal layer from an anterior to a posterior of a heart. In some example embodiments, alone or in combination with any of the previous example embodiments, the at least one incision length is made in the parietal layer from a posterior base to an apex of a heart. In some example embodiments, alone or in combination with any of the previous example embodiments, the at least one incision length is made in the parietal layer from a posterior right atrium to an apex of a heart. In some example embodiments, alone or in combination with any of the previous example embodiments, the at least one incision length is made in the parietal layer from a left ascending aorta to an apex of a heart. In some example embodiments, alone or in combination with any of the previous example embodiments, the at least one incision length is made in the parietal layer from a right ascending aorta to an apex of a heart. In some example embodiments, alone or in combination with any of the previous example embodiments, the at least one incision length is made in the parietal layer transversely about a heart.

In some example embodiments, alone or in combination with any of the previous example embodiments, the method also includes, before creating the incision length, puncturing pericardial tissue and providing an access point into a pericardial space. In some example embodiments, alone or in combination with any of the previous example embodiments, the method also includes, before puncturing, providing subxiphoid access to the pericardium. In some example embodiments, alone or in combination with any of the previous example embodiments, the method also includes, before puncturing, providing transvascular access to the pericardium. In some example embodiments, alone or in combination with any of the previous example embodiments, the method also includes moving the blade from the deployed position to the retracted position before removing the incision device from the pericardium.

In some example embodiments, alone or in combination with any of the previous example embodiments, the method also includes puncturing into the pericardium via an introducer. In some example embodiments, alone or in combination with any of the previous example embodiments, the method also includes, after puncturing, inserting a guidewire into the pericardial space. In some example embodiments, alone or in combination with any of the previous example embodiments, the method also includes stabilizing the incision device within the pericardium. In some example embodiments, alone or in combination with any of the previous example embodiments, the incision device is stabilized via one or more stabilization member. In some example embodiments, alone or in combination with any of the previous example embodiments, the one or more stabilizing members are structured to move between a stabilizing retracted position and a stabilizing deployed position with the one or more stabilizing members being moved from the stabilizing retracted position to the stabilizing deployed position upon positioning of the multi-lumen catheter within the human body.

In some example embodiments, alone or in combination with any of the previous example embodiments, the one or more stabilizing member is a wire, loop, or shape-memory metal. In some example embodiments, alone or in combination with any of the previous example embodiments, the one or more stabilizing member is one or more inflatable structures. In some example embodiments, alone or in combination with any of the previous example embodiments, the method also includes moving the stabilizing member from the stabilizing deployed position to the stabilizing retracted position upon an initial puncture is made into the pericardium by the blade.

In some example embodiments, alone or in combination with any of the previous example embodiments, the method also includes moving a sheath from a first position covering the incision channel to a second position that uncovers the incision channel. In some example embodiments, alone or in combination with any of the previous example embodiments, the sheath is structured to cover the incision channel in an instance in which the multi-lumen catheter is not positioned within human body. In some example embodiments, alone or in combination with any of the previous example embodiments, the one or more openings of the sheath are traversable along the multi-lumen catheter allowing an actuator to cause the one or more stabilizing members to laterally project through both the multi-lumen catheter and the sheath.

In some example embodiments, alone or in combination with any of the previous example embodiments, the method includes sterilizing the incision device. In some example embodiments, alone or in combination with any of the previous example embodiments, the incision device is sterilized via E-beam sterilization. In some example embodiments, alone or in combination with any of the previous example embodiments, the incision device is sterilized via gamma sterilization. In some example embodiments, alone or in combination with any of the previous example embodiments, the incision device is sterilized via ethylene oxide sterilization. In some example embodiments, alone or in combination with any of the previous example embodiments, the incision device is sterilized via autoclave sterilization.

In some example embodiments, alone or in combination with any of the previous example embodiments, the method also includes determining a location of the incision device within the pericardium via ultrasound. In some example embodiments, alone or in combination with any of the previous example embodiments, creating the at least one incision length is determined in response to a signal indicative of a reduction of restraint of the heart. In some example embodiments, alone or in combination with any of the previous example embodiments, creating the at least one incision length is determined in response to a signal indicative of a reduction of restraint of the heart; and repeating the creating of the at least one incision length.

In some example embodiments, alone or in combination with any of the previous example embodiments, the method also includes, after creating the at least one incision length, confirming a location of a distal end of the multi-lumen catheter device; and in response to a signal indicative of a reduction of restraint of the heart, repeating the steps of creating the at least one incision length, and confirming a location of the distal end.

In some example embodiments, alone or in combination with any of the previous example embodiments, at least a portion of the incision device includes an electrode. In some example embodiments, alone or in combination with any of the previous example embodiments, at least a portion of the cutting surface includes an electrode. In some example embodiments, alone or in combination with any of the previous example embodiments, the method also includes providing an electrical current to the electrode. In some example embodiments, alone or in combination with any of the previous example embodiments, the electrode is a wire. In some example embodiments, alone or in combination with any of the previous example embodiments, the wire is shaped as one or more arcs projecting laterally through the multi-lumen catheter along the longitudinal axis.

In some example embodiments, alone or in combination with any of the previous example embodiments, the cutting surface includes a scalpel. In some example embodiments, alone or in combination with any of the previous example embodiments, at least a portion of the scalpel includes an electrode. In some example embodiments, alone or in combination with any of the previous example embodiments, the at least one electrode is electrically couplable to a source of radiofrequency energy or electrical current sufficient to cut, separate, scissor, or evaporate a portion of the parietal layer.

In some example embodiments, alone or in combination with any of the previous example embodiments, the method also includes receiving a signal from at least one nerve detection device. In some example embodiments, alone or in combination with any of the previous example embodiments, the at least one nerve detection device is located on the multi-lumen catheter. In some example embodiments, alone or in combination with any of the previous example embodiments, the at least one nerve detection device is located adjacent the incision device. In some example embodiments, alone or in combination with any of the previous example embodiments, the at least one nerve detection device is located on the puncturing tip. In some example embodiments, alone or in combination with any of the previous example embodiments, the at least one nerve detection device is located on the cutting surface.

In some example embodiments, alone or in combination with any of the previous example embodiments, the method also includes receiving a signal from at least one nerve stimulation device. In some example embodiments, alone or in combination with any of the previous example embodiments, the at least one nerve stimulation device is located on the multi-lumen catheter. In some example embodiments, alone or in combination with any of the previous example embodiments, the at least one nerve stimulation device is located adjacent the incision device. In some example embodiments, alone or in combination with any of the previous example embodiments, the at least one nerve stimulation device is located on the puncturing tip. In some example embodiments, alone or in combination with any of the previous example embodiments, the at least one nerve stimulation device is located on the cutting surface.

In another example embodiment, a medical device is provided. The medical device includes an incision device structured to be operably coupled to a distal end of a catheter. The incision device includes an incision channel defined along the incision device. The medical device also includes a retractable cutting apparatus disposed within the incision channel. The retractable cutting apparatus includes a gear-actuated blade structured to move between a retracted position and a deployed position within the incision channel. In an instance in which the gear-actuated blade is in the retracted position, the gear-actuated blade is disposed within the incision channel. In an instance in which the gear-actuated blade is in the deployed position, the gear-actuated blade at least partially protrudes from the incision channel. The retractable cutting apparatus also includes a torsion gear mechanism attached to the gear-actuated blade. The torsion gear is structured to move the gear-actuated blade between the retracted position and the deployed position. The retractable cutting apparatus further includes a rotatable gear actuator that is structured to move the torsion gear mechanism.

In some example embodiments, alone or in combination with any of the previous example embodiments, the retractable cutting apparatus includes a force transferring gear mechanism.

In some example embodiments, alone or in combination with any of the previous example embodiments, the retractable cutting apparatus including a force transferring gear mechanism in the form of a worm gear.

In some example embodiments, alone or in combination with any of the previous example embodiments, the retractable cutting apparatus includes an intermediate actuation member with the intermediate actuation member being disposed between the force transferring gear mechanism and the torsion gear mechanism.

In some example embodiments, alone or in combination with any of the previous example embodiments, the torsion gear mechanism is a rack and pinion gear.

In some example embodiments, alone or in combination with any of the previous example embodiments, a rack portion of the rack and pinion gear is attached to the gear-actuated blade.

In some example embodiments, alone or in combination with any of the previous example embodiments, the blade is structured to move along a latitudinal axis upon actuation of the torsion gear mechanism.

In some example embodiments, alone or in combination with any of the previous example embodiments, a pinion portion of the rack is a pinion gear is attached to the rotatable gear actuator with the rotation of the rotatable gear actuator causing the rack portion to move along the pinion portion of the rack and pinion gear.

In some example embodiments, alone or in combination with any of the previous example embodiments, a pinion portion of the rack and pinion gear is attached to the intermediate actuation member.

In some example embodiments, alone or in combination with any of the previous example embodiments, the intermediate actuation member defines a proximal end and a distal end. In such an embodiment, at least a portion of the force transferring gear mechanism is attached to the proximal end of the intermediate actuation member and at least a portion of the torsion gear mechanism is attached to the distal end of the intermediate actuation member.

In some example embodiments, alone or in combination with any of the previous example embodiments, one or more stabilizing members are provided along the incision device with the one or more stabilizing members being structured to maintain the incision device in place during an incision by the gear-actuated blade.

In some example embodiments, alone or in combination with any of the previous example embodiments, the gear-actuated blade comprises a cutting surface.

In some example embodiments, alone or in combination with any of the previous example embodiments, at least a portion of cutting surface is reversibly adjustable laterally relative to the longitudinal axis of the multi-lumen catheter between a range of angles.

In some example embodiments, alone or in combination with any of the previous example embodiments, at least a portion of the retractable cutting apparatus or the incision device is radiopaque.

In some example embodiments, alone or in combination with any of the previous example embodiments, the medical device is sterilized.

In some example embodiments, alone or in combination with any of the previous example embodiments, the medical device also includes an introducer positioned near the distal end of the catheter.

In some example embodiments, alone or in combination with any of the previous example embodiments, at least a portion of the incision device or retractable cutting apparatus comprises an electrode.

In some example embodiments, alone or in combination with any of the previous example embodiments, the medical device includes a controller engaged to the incision device.

In some example embodiments, alone or in combination with any of the previous example embodiments, the controller is structured to provide at least one of movement of the rotatable gear actuator, movement of the stabilizing member(s) between a retracted position and a deployed position, movement of the incision device, movement of the catheter, or energy to the incision device.

In another example embodiment, a method of manipulating a transection device is provided. The method includes providing a medical device of any one of the example embodiments herein engaged with a controller. The method also includes controlling at least one of movement of the rotatable gear actuator, movement of the one or more stabilizing members, movement of the incision device, movement of the catheter, or supplying energy to the incision device.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIG. 1C is a further enlarged view of section 1C of FIG. 1A depicting the serosal, visceral, fibrous layers and adipose tissue of the parietal pericardium, including the pericardial cavity.

FIG. 2A is a medical device of an example embodiment with a sheath covering an incision channel in accordance with various embodiments.

FIG. 2B is a medical device of an example embodiment in the deployed position with the sheath uncovered in accordance with various embodiments.

FIG. 3A illustrates an example retractable cutting apparatus in the retracted position in accordance with various embodiments.

FIG. 3B illustrates the example retractable cutting apparatus of FIG. 3A in the deployed position in accordance with various embodiments.

FIG. 3C is another view of the example retractable cutting apparatus shown in FIGS. 3A and 3B in accordance with various embodiments.

FIG. 4A illustrates another example retractable cutting apparatus in the retracted position in accordance with various embodiments.

FIG. 4B illustrates the example retractable cutting apparatus of FIG. 4A in the deployed position in accordance with various embodiments.

FIGS. 5A-5C illustrate the medical device being deployed within the pericardium in accordance with various embodiments.

FIG. 6 depicts an exemplary controller device for delivering the incision devices in accordance with various embodiments.

FIG. 7 illustrates a medical device of various embodiments positioned within the pericardial cavity of a human in accordance with various embodiments.

FIG. 8 illustrates another example medical device with stabilizing members in accordance with various embodiments.

FIG. 9 is a simplified diagram of a multi-lumen catheter approach to the pericardial cavity in accordance with various embodiments.

FIG. 10 is a simplified diagram of an alternative multi-lumen catheter approach to the pericardial cavity in accordance with various embodiments.

FIG. 11 is a simplified diagram of a parietal layer incision length and cut path in accordance with various embodiments.

### DETAILED DESCRIPTION

The present disclosure provides for a catheter-based therapy referred to as transcatheter alleviation of pericardial restraint (TAPR) that can reduce pericardial restraint by incising or opening the pericardium with the intention of improving patient outcomes with heart dysfunction, for example, HFpEF or HFrEF and reducing HF readmissions related thereto. The present disclosure, in one example, provides a device with a concealed/medially-facing cutting surface for accessing and modifying a subject's pericardium for relieving pericardial restraint and/or resolving a heart dysfunction. The present disclosure further provides for methods of treating heart dysfunction using the presently disclosed device.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

As used herein the phrase “cutting surface” is inclusive of one or more of an edge of a sharpened blade or the surface of an electrode structured to receive sufficient current or radio frequency energy (RF) to ablate, burn, vaporize, or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a multi-lumen catheter device or catheter, and the application of a directional force sufficient to cut or separate the tissue, the force being substantially in a direction towards the proximal end of the multi-lumen catheter device, for example, by pulling the multi-lumen catheter device while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and the like.

As used herein the phrase “incision length” is inclusive of a non-zero distance of a cut or incision, for example, beginning at a first point, e.g., a target point, and terminating at a second point, e.g., an access point. An incision length can be linear, non-linear, or a plurality of linear and/or non-linear lengths that intersect or do not intersect about a curved or non-planar surface, such a heart.

As used herein the phrase “reducing pressure” and “reducing restraint” are inclusive of their ordinary and customary meaning of one to ordinary skill in medical and surgical arts.

As used herein the phrase “preserved ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “reduced ejection fraction” is inclusive of the ordinary and customary meaning to one of ordinary skill in medical and surgical arts, for example, impairment of ventricular filling or ejection of blood or both, with a clinical syndrome in which patients display left ventricular ejection fraction (LVEF) of 40% or less and are accompanied by progressive left ventricular dilatation and adverse cardiac remodeling and/or mitral valve dysfunction.

As used herein the phrase “heart dysfunction” is inclusive of the ordinary and customary meaning to one of ordinary skill in medical and surgical arts, for example, heart failure or congestive heart failure.

As used herein the phrase “incision device” is inclusive of a device with a cutting surface, for example an edge of a blade or a surface of an electrode.

As used herein the phrase “pericardial incision assembly” and “incision assembly” are used interchangeable and refer to an assemblage that includes an incision device.

As used herein the phrase “multi-lumen catheter device” is inclusive of a catheter structured with at least two lumens comprising a medical instrument, device, or component thereof, for example, an incision device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein the phrase “puncturing tip” is inclusive of an atraumatic object suitable for puncturing or penetrating tissue without substantial trauma to or bleeding from the vicinity of the picture or penetration.

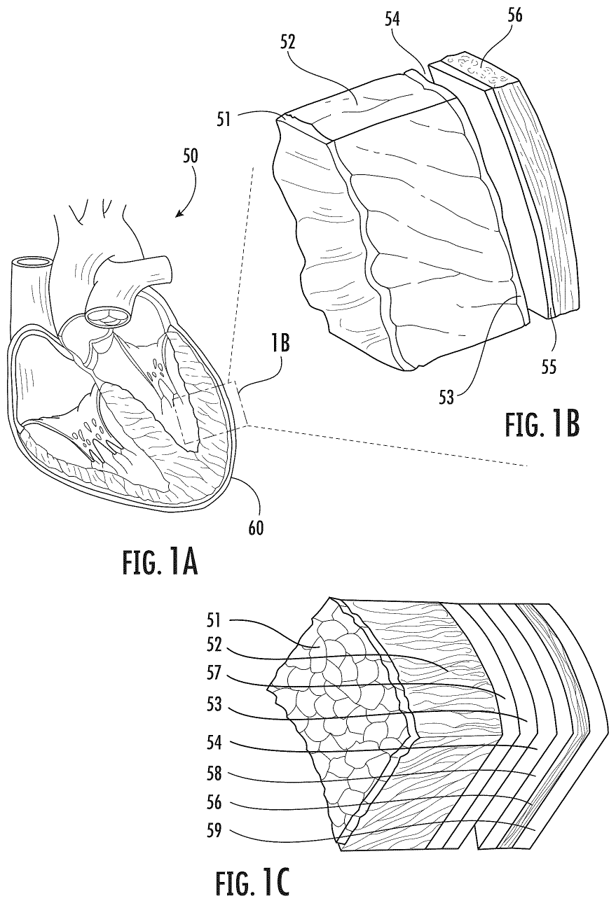

With reference to FIGS. 1A, 1B, 1C, and sections 1B, 1C, layers of a heart wall of a heart 50, with pericardium 60, from inside-out, being the endocardium 51, the myocardium 52, epicardial adipose tissue 57, the visceral layer 53 of the pericardium, the pericardial cavity 54, the parietal layer 55 of the pericardium, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity and superficial to the visceral layer 53 of the pericardium 60.

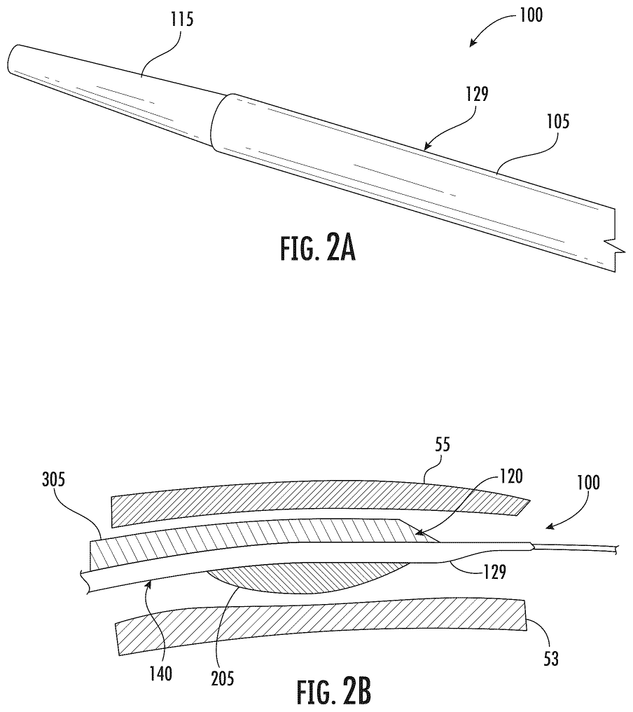

Referring now to FIGS. 2A and 2B, in one example the presently disclosed medical device 100 comprises a flexible catheter 129 comprising a distal end, at least one lumen, and a longitudinal axis; an incision device 140 coupled to the distal end of the catheter; and an introducer 115 coupled to and projecting from the incision device 140. The catheter may be steerable. In one example, the introducer 115 comprises a puncturing tip. The puncturing tip of the introducer 115 is structured to provide enough force to the introducer to puncture human tissue to allow access to the pericardial cavity.

In one example, at least a portion of the flexible catheter 129 tip is radiopaque. In one example, at least a portion of the incision device 140 is radiopaque. In one example, at least a portion of the introducer 115 tip is radiopaque.

As shown in FIG. 2A, the medical device 100 may also include a sheath 105 structured to moveably cover the incision channel 120. The sheath 105 is structured to cover the incision channel 120 in an instance in which the multi-lumen catheter 129 is not positioned within human body. Once the multi-lumen catheter has been positioned (e.g., the incision device is positioned where the cut in the parietal layer of the pericardium is to begin), the sheath 105 may be actuated to uncover the incision channel 120. In some example embodiments, alone or in combination with any of the previous example embodiments, the sheath 105 may not be moveable and merely has a slit to allow the knife to protrude. The sheath 105 can also hold the one or more stabilizing members in the stabilizing retracted position during movement (e.g., the sheath moves to uncover the stabilizing member(s) upon placement in the pericardial cavity).

The incision device 140 defines an incision channel 120 that is structured to receive a retractable cutting apparatus 300. The retractable cutting apparatus 300 can be moved along the incision channel 120 via an internal actuator (e.g., connected to the rotatable gear actuator 315) to move the retractable cutting apparatus 300 between a deployed position and a retracted position.

FIG. 2B illustrates the blade of the retractable cutting apparatus 300 in the deployed position. As discussed herein, the blade is structured to move between a retracted position and a deployed position. In the retracted position, the blade is disposed within the incision channel (e.g., not protruding from the incision channel). In the deployed position, the blade at least partially protrudes from the incision channel. As discussed herein in more detail, the device 100 may have one or more stabilizing members 205 that are structured to maintain the position of the incision device 140 when deployed.

Part or all of the medical device 100 may be sterilized for use. The medical device may be sterilized using various sterilizing techniques, such as E-Beam sterilization, gamma sterilization, ethylene oxide sterilization, autoclave sterilization, including aseptic sterilization and aseptic manufacturing/packaging, and/or the like. Additionally, one or more materials used in the medical device may have anti-bacterial characteristics. The pericardial transection device and/or catheter and/or sheath can be structured such that the total outer diameter (O.D.) introduced to the pericardial cavity is between about 6 Fr (2 mm) and about 30 Fr (10 mm). The pericardial transection device and/or catheter and/or sheath can be structured such that the total outer diameter (O.D.) introduced to the pericardial cavity is between about 6 Fr (2 mm) and about 20 Fr (6.67 mm). The pericardial transection device and/or catheter and/or sheath can be structured such that the total outer diameter (O.D.) introduced to the pericardial cavity is between about 6 Fr (2 mm) and about 15 Fr (5 mm). The pericardial transection device and/or catheter and/or sheath can be structured such that the total outer diameter (O.D.) introduced to the pericardial cavity is between about 6 Fr (2 mm) and about 12 Fr (4 mm). The pericardial transection device and/or catheter and/or sheath can be structured such that the total outer diameter (O.D.) introduced to the pericardial cavity is approximately 10 Fr (3.33 mm).

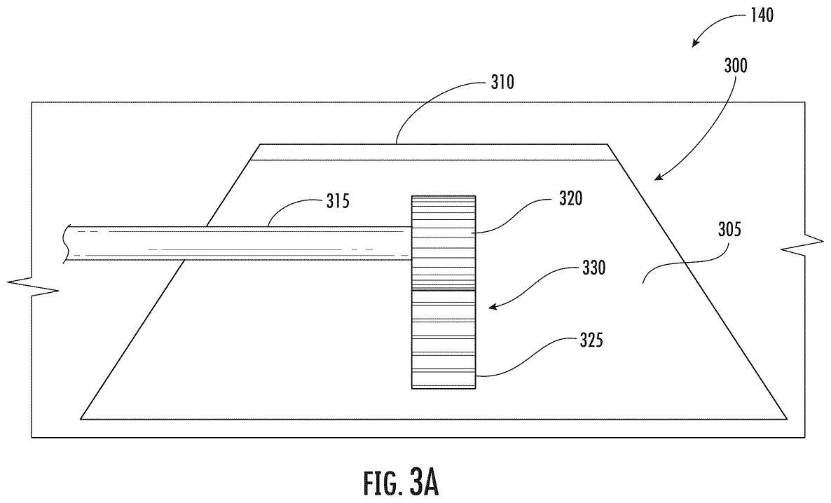

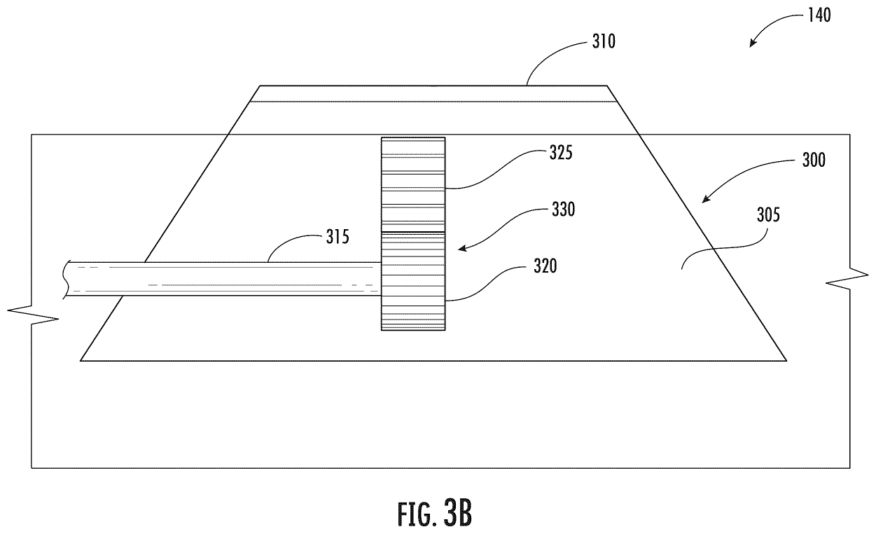

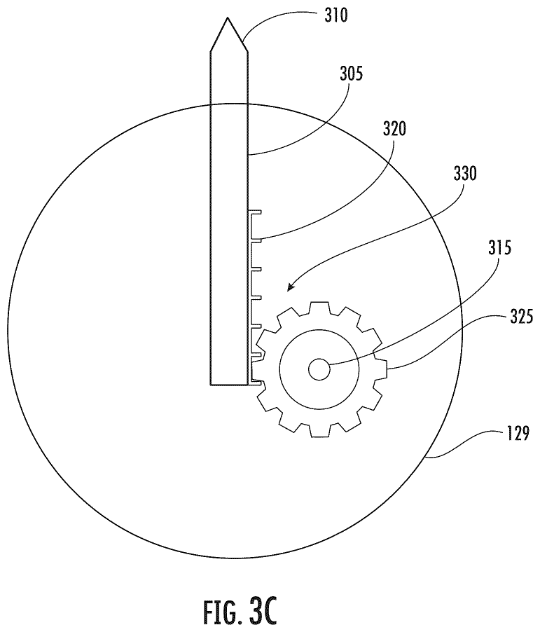

Referring now to FIGS. 3A-3C, an example retractable cutting apparatus 300 is shown. As discussed above in reference to FIGS. 2A and 2B, the retractable cutting apparatus 300 is movably positioned within the incision channel 120 of the incision device between a retracted position (FIG. 3A) and a deployed position (FIG. 3B). FIG. 3A illustrates an example retractable cutting apparatus 300 in the retracted position. FIG. 3B illustrates the retractable cutting apparatus 300 in the deployed position. FIG. 3C illustrates another view of the retractable cutting apparatus 300 in the deployed position (e.g., from the perspective along the catheter).

The retractable cutting apparatus 300 includes a blade 305, a torsion gear mechanism 330, and a rotatable gear actuator 315. The rotatable gear actuator 315 is rotatable to cause the torsion gear mechanism 330 to move the blade 305 along the latitudinal axis between a retracted position and a deployed position. As shown in FIG. 3A, the blade 305 is disposed within the multi-lumen catheter in the retracted position and then via the movement of the rotatable gear actuator 315 is moved to protrude from the multi-lumen catheter, as shown in FIG. 3B.

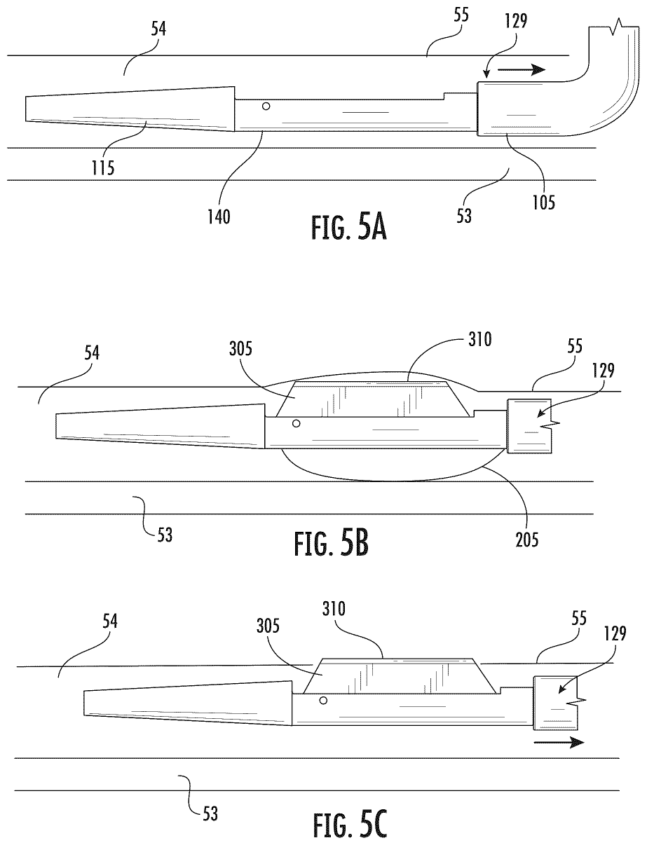

The blade 305 may have various shapes based on the desired incision length and/or force provided to make the incision. As shown, the blade 305 may define a cutting surface 310 that is positioned to cut into the parietal layer of the pericardium, as discussed in more detail in reference to FIGS. 5A-5C below. In some instances, the blade 305 may have one or more puncturing tips to assist the blade 305 in making the initial incision into the parietal layer of the pericardium. The blade 305 may use an electrode to cause the incision. Additionally or alternatively, the cutting surface 310 may be a sharp edge to mechanically cut the parietal layer of pericardium. As shown in FIG. 3A, in the retracted position, the blade 305 is fully disposed within the incision device 140. As such, the blade 305 may not be exposed outside of the multi-lumen catheter, allowing for the incision device 140 to be safely positioned within the pericardial cavity for use.

The blade 305 may have a cutting surface 310 that is positioned to cut tissue upon being moved within the human body in an instance in which the blade 305 is in the deployed position. The cutting surface 310 of the blade 305 when laterally projected (e.g., in the deployed position), is approximately parallel to the longitudinal axis of the multi-lumen catheter. In some example embodiments, alone or in combination with any of the previous example embodiments, the cutting surface 310 of the blade 305 when laterally projected, faces away from the distal end and towards the proximal end of the multi-lumen catheter. The blade 305 is structured to have a sufficient force to puncture pericardial tissue (e.g., the parietal layer) in an instance in which the retractable cutting apparatus 300 is moved from the retracted position to the deployed position.

Upon positioning of the incision device 140 within the pericardial cavity, the blade 305 is moved via the rotatable gear actuator 315 from the retracted position to the deployed position as shown in FIG. 3B. The deployed position may be any position in which at least a portion of the blade 305 protrudes from the incision channel (of the incision device 140). As such, the blade 305 may define multiple deployed positions along the torsion gear mechanism that expose a different amount of the blade 305. For example, the size of an individual's pericardial cavity may vary, causing less or more blade to be exposed (e.g., protrude from the incision channel) in order to perform the incision. As shown in FIGS. 3A and 3B, the blade 305 is fixed along the longitudinal axis, such that the blade 305 only moves along the latitudinal axis.

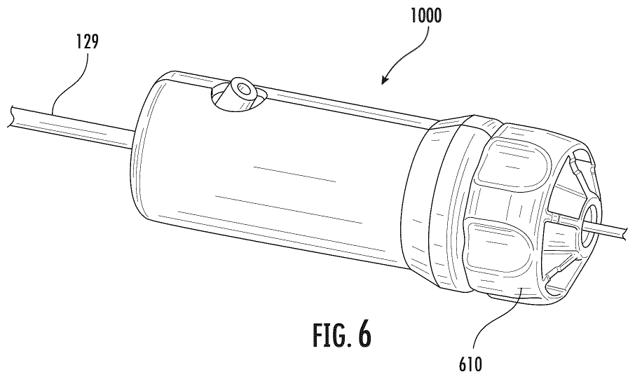

The rotatable gear actuator 315 is rotatably fixed along the longitudinal axis. The rotatable gear actuator 315 may be a guide member that is received by a guide member lumen of the multi-lumen catheter. The guide member may extend through the given lumen of the multi-lumen catheter and extend into the controller of the multi-lumen catheter as discussed herein. The controller may include a guide member movement actuator (e.g., knob 610) that is attached to the guide member. The guide member movement actuator is manipulated to rotate the guide member. The rotatable gear actuator 315 can be manipulated to move the torsion gear mechanism 330 (and subsequently the blade 305). The rotatable gear actuator 315 may be manipulated by a controller (e.g., a knob 610 shown in FIG. 6).

The torsion gear mechanism 330 may be a rack and pinion gear assembly defining a rack portion 325 and a pinion portion 320. The pinion portion 320 of the torsion gear mechanism 330 may be attached to the rotatable gear actuator 315. The pinion portion 320 may be fixed by the rotatable gear actuator 315, such that the pinion portion 320 is structured to rotate based on the rotation of the rotatable gear actuator 315. The rotation of the pinion portion 320 causes the rack portion 325 to translate along the latitudinal axis. The rack portion 325 of the torsion gear mechanism 330 is fixed to the blade 305, such that the blade 305 moves along the latitudinal axis based on the movement of the torsion gear mechanism 330. The rack portion 325 of the torsion gear mechanism 330 may be formed out of a plurality of notches in the blade itself (e.g., the pinion portion engages with the notches in the blade). The torsion gear mechanism 330 may be structured to maintain the engagement of the rack portion 325 and a pinion portion 320 during operation.

As the pinion portion 320 rotates in the clockwise direction (from the perspective of the proximal end of the multi-lumen catheter), the blade 305 is structured to move from the retracted position to the deployed position. Alternatively, as the pinion portion 320 rotates in the counterclockwise direction (from the perspective of the proximal end of the multi-lumen catheter), the blade 305 is structured to move from the deployed position to the retracted position.

Upon moving the retractable cutting apparatus 300 into the deployed position, the multi-lumen catheter is moveable along the pericardium to allow an incision to be made in the parietal layer. The incision may define an incision length that is defined as the length in which the multi-lumen catheter is moved in an instance in which the retractable cutting apparatus 300 is in the deployed position. The incision may be linear due to the movement of the multi-lumen catheter. The retractable cutting apparatus 300 remains generally stationary relative to the multi-lumen catheter while the multi-lumen catheter is moving. Upon completing the incision, the blade 305 is moved from the deployed position to the retracted position to be removed from the human.

One or more components of the medical device may be radiopaque to allow the medical device to be seen within the human body using ultrasound. In various embodiments, portions of the retractable cutting apparatus, multi-lumen catheter, and/or introducer may be radiopaque.

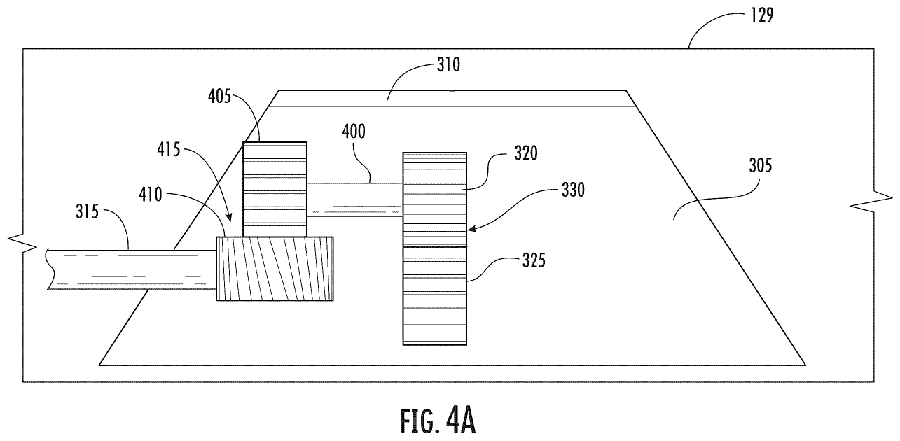

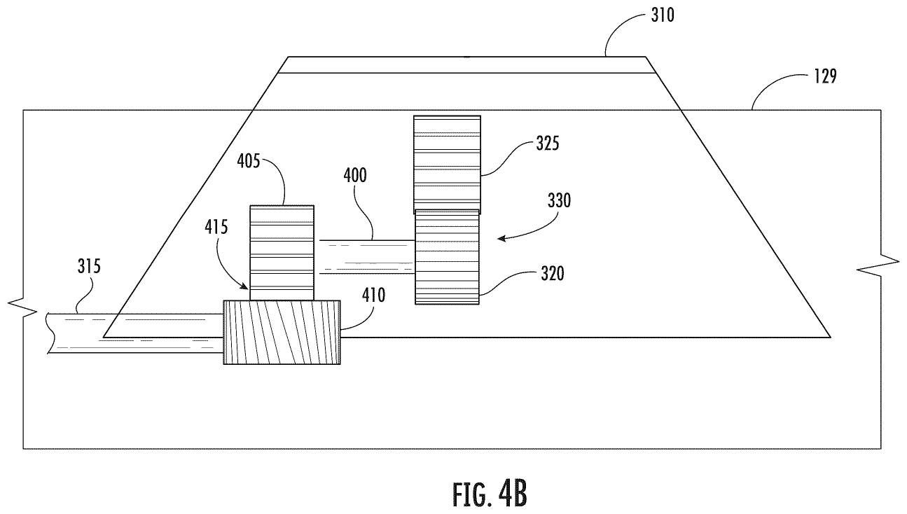

Referring now to FIGS. 4A-4B, another example retractable cutting apparatus 300 is shown. As discussed above in reference to FIGS. 2A and 2B, the retractable cutting apparatus 300 is movably positioned within the incision channel 120 of the incision device between a retracted position (FIG. 4A) and a deployed position (FIG. 4B). FIG. 4A illustrates an example retractable cutting apparatus 300 in the retracted position. FIG. 4B illustrates the retractable cutting apparatus 300 in the deployed position.

The embodiment shown in FIGS. 4A and 4B includes an intermediate actuation member 400 that is positioned between the rotatable gear actuator 315 and the torsion gear mechanism 330. The intermediate actuation member 400 is generally parallel to the rotatable gear actuator 315. The intermediate actuation member 400 may be attached to the torsion gear mechanism 330 and a force transferring gear mechanism 415.

The torsion gear mechanism 330 may be the same and operate the same as the torsion gear mechanism 330 of FIGS. 3A and 3B (e.g., a rack and pinion gear). However, instead of the pinion portion 320 being directly attached to the rotatable gear actuator 315, the pinion portion 320 is attached to the intermediate actuation member 400. The intermediate actuation member 400 is caused to rotate by rotation of the rotatable gear actuator 315, which also causes the attached pinion portion 320 to rotate. As such, the pinion portion 320 is still rotated based on manipulation of the rotatable gear actuator 315.

The rotational force may be transferred from the rotatable gear actuator 315 to the intermediate actuation member 400 via the force transferring gear mechanism 415. The force transferring gear mechanism 415 may be a worm gear assembly that is structured to transfer the rotational force from the rotatable gear actuator 315 to the intermediate actuation member 400. The actuator portion 410 may be a spiral threaded shaft (worm gear) either attached to or unitary with the rotatable gear actuator 315. The rotation of the rotatable gear actuator 315 causes the spiral threaded shaft to engage with the gear 405 (worm wheel) attached to the intermediate actuation member 400. The intermediate actuation member 400 is caused to rotate based on the engagement of the force transferring gear mechanism 415. The gear 405 is attached to a proximal end of the intermediate actuation member 400 and the pinion portion 320 is attached to a distal end of the intermediate actuation member 400, such that the rotation of the gear 405 (caused by the rotation of the actuator portion 410) causes the intermediate actuation member 400 and the pinion portion 320 to rotate.

As the pinion portion 320 rotates in the clockwise direction (from the perspective of the proximal end of the multi-lumen catheter), the blade 305 is structured to move from the retracted position to the deployed position. Alternatively, as the pinion portion 320 rotates in the counterclockwise direction (from the perspective of the proximal end of the multi-lumen catheter), the blade 305 is structured to move from the deployed position to the retracted position.

Upon moving the retractable cutting apparatus 300 into the deployed position, the multi-lumen catheter is moveable along the pericardium to allow an incision to be made in parietal layer. The incision may define an incision length that is defined as the length in which the multi-lumen catheter is moved in an instance in which the retractable cutting apparatus 300 is in the deployed position. The incision may be linear due to the movement of the multi-lumen catheter. The retractable cutting apparatus 300 remains generally stationary relative to the multi-lumen catheter while the multi-lumen catheter is moving. Upon completing the incision, the blade 305 is moved from the deployed position to the retracted position to be removed from the human.

One or more components of the medical device may be radiopaque to allow the medical device to be seen within the human body using ultrasound. In various embodiments, portions of the retractable cutting apparatus, multi-lumen catheter, and/or introducer may be radiopaque.

FIGS. 5A-5C illustrate medical device 100 being deployed. In one example, device 100 is deployed subxiphoidally, e.g., through percutaneous access achieved by directing a needle into the patient under the xiphoid process towards the pericardial cavity. In another example, device 100 is deployed intravascularly thru the right ventricle. The medical device 100 can be advanced through a subject's pericardial cavity 54 in undeployed configuration with the blade 305 in the retracted position within the incision channel. The initial access to the pericardial cavity 54 may be achieved via the introducer 115. As the medical device is positioned within the pericardial cavity 54, the sheath 105 may be covering the incision channel. As the multi-lumen catheter is positioned within the pericardial cavity 54 at the location of the incision, the sheath 105 may be removed, as shown in FIG. 5A. The sheath may uncover the incision device 140 that is still in the retracted position.

As shown in FIG. 5B, the medical device 100 may have one or more stabilizing members 205. The stabilizing member(s) 205 may be independently user controlled by advancing an actuating wire distally toward introducer 115 tip that laterally extends stabilizing member(s) 205 a distance from medical device 100. In one example, two or more stabilizing members 205 are positioned radially about the assembly. In one example, two or more stabilizing members 205 are positioned radially about the assembly about 120 degrees apart. In one example, the two or more stabilizing members 205 are offset longitudinally from the cutting surface to minimize or eliminate pushing the device through the newly cut slit in the parietal layer of the pericardium just as it is formed. Stabilizing members 205 can be flexible rods or strip, or inflatable structures, such as balloons that can be inflated with air or liquid (saline).

The one or more stabilizing members 205 are structured to move between a stabilizing retracted position and a stabilizing deployed position upon positioning of the multi-lumen catheter within the human body.

The one or more stabilizing members 205 may be moved to the stabilizing retracted position after the blade has punctured the parietal layer 55. For example, the stabilizing member(s) 205 may be structured to deploy to stabilize the multi-lumen catheter while the blade is making the cut into the tissue. After the initial cut into the parietal layer 55 has been made, the stabilizing member(s) 205 may be moved to the stabilizing retracted position to allow the multi-lumen catheter to be moved along the pericardial cavity.

The one or more stabilizing members 205 may be a stabilizing balloon. The one or more stabilizing members 205 may be a stabilizing balloon made out of a polymer material. The one or more stabilizing members 205 may be a stabilizing balloon to be filled with saline (e.g., by a syringe). The one or more stabilizing balloons may be positioned to contact an epicardium upon positioning of the medical device within a pericardial cavity. In various embodiments, the blade 305 is longer in a longitudinal direction that the stabilizing balloon in the longitudinal direction. The one or more stabilizing members 205 and the blade 305 may be independently adjusted. The one or more stabilizing members 205 and/or the blade 305 may be adjusted within the human. The adjustment of the one or more stabilizing members 205 and/or the blade 305 may be based on the size of the pericardial cavity of the human.

The retractable cutting apparatus 300 may be moved from the retracted position to the deployed position as discussed herein. The retractable cutting apparatus 300 may use the puncture tip of the blade 305 to puncture the pericardial tissue to be cut. The retractable cutting apparatus 300 may use the sheer force of the blade 305 to cause the incision. After the blade 305 punctures the pericardial tissue, the incision can be made along the pericardium tissue. In an instance in which the medical device 100 has stabilizing member(s) 205, the stabilizing member(s) 205 may be moved to the stabilizing retracted position to allow for the multi-lumen catheter to be moved. A portion of the blade 305 can serve as a radiopaque marker indicating ‘bunching up’ of tissue, for example, due to excessive pull before a through-cut is achieved. In one example, the back silhouette of the blade may be selectively insulated (as it may be activated to achieve the exit puncture) to avoid ablation damage to neighboring anatomy.

Upon puncture of the pericardium tissue by the blade 305, the multi-lumen catheter 129 is moved along the pericardial cavity 54 to cause the incision in the parietal layer for the incision length intended. The cutting surface 310 of the blade 305 cause the incision. The cutting surface 310 of the blade 305 may be parallel to the longitudinal axis of the catheter. Alternatively, the cutting surface 310 of the blade 305 may have an incline, such that the cutting surface is facing the proximal end of the catheter. In some instances, the incision is a linear incision along the pericardium tissue. Alternatively, the incision may be non-linear. Upon moving the multi-lumen catheter the length of the intended incision, the retractable cutting apparatus is moved back to the retracted position and the sheath 105 can be moved to cover the incision channel. The multi-lumen catheter can then be removed from the human body without having any blades exposed.

In one example, at least one incision length is made in the parietal layer of the pericardium of a heart. The at least one incision lengths, in a heart with a dysfunction treatable with the present method, may cause the parietal layer of the pericardium to separate radially about the cut line, without the need for removal of pericardial tissue. Other incision lengths and paths may be employed. Combinations of incision lengths and paths, and combination of incision lengths and paths with one or more of partial removal of pericardium, drainage, and other pericardial treatments can be employed.

In one example, creating at least one incision length is determined in response to a signal indicative of a reduction of restraint of the heart. In one example, creating at least one incision length is determined in response to a signal indicative of a reduction of restraint of the heart; and repeating the creating of at least one incision length. In one example, the presently disclosed method further comprises, after creating the at least one incision length, confirming a location of a distal end of the multi-lumen catheter device; and in response to a signal indicative of a reduction of restraint of the heart, repeating the steps of creating the at least one incision length, and confirming a location of the distal end.

FIG. 6 illustrates a controller 1000 with a guide member movement actuator (e.g., actuating knob 610) that is structured to rotate the rotatable gear actuator 315. The controller 1000 may be a handle. The guide member movement actuator 610 may have an internal fixing point that fixably attaches the rotatable gear actuator 315 to the knob 610. As the knob 610 is rotated, the rotatable gear actuator 315 is also caused to rotate in the same direction as the knob 610 is rotated. As such, when the knob 610 is moved in the clockwise direction, the retractable cutting apparatus 300 is moved from the retracted position to the deployed position. In an instance in which the knob 610 is moved in the counterclockwise direction, the retractable cutting apparatus 300 is moved from the deployed position to the retracted position. The rotation of the knob 610 and subsequently the rotatable gear actuator 315 causes the blade to move via the torsion gear mechanism 330 (e.g., rack and pinion gear). For example, the pinion portion 320 is caused to rotate casing the rack portion 325 attached to the blade to move relative to the pinion portion 320, causing the blade to move along the latitudinal axis between the retracted position and the deployed position. The rack portion 325 may also be one or more notches in the blade 305 receive the pinion portion 320 during rotation. The controller 1000 may have various other capabilities not shown (e.g., the controller may have multiple actuating knobs and/or actuating buttons for controlling the multi-lumen catheter 129 and the various components of the medical device 100).

A method of manipulating medical device 100 would include providing medical device 100 engaged with controller 1000 and controlling, for example, manipulating any one or combination of multiple actuating knob 610 or any other knobs provided on the controller 1000 to provide at least one of movement of the rotatable gear actuator, movement of the one or more stabilizing members, movement of the incision device, movement of the catheter, or energy supply to the incision device with controller 1000. The aforementioned method is applicable to any previously disclosed transection devices.

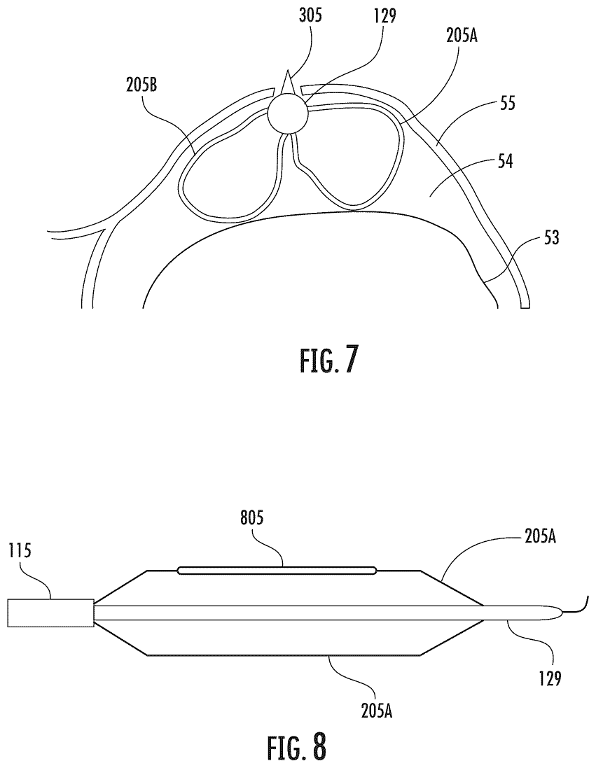

FIG. 7 illustrates a medical device of various embodiments positioned within the pericardial cavity of a human in accordance with various embodiments. As shown, the medical device includes a first stabilizing member 205A and a second stabilizing member 205B. The first stabilizing member 205A and the second stabilizing member 205B may expand to contact the walls of the pericardial cavity 54 to allow for the blade 305 to puncture the parietal layer 55. The first stabilizing member 205A and the second stabilizing member 205B may contact both the parietal layer 55 and the visceral layer 53 when deployed. The first stabilizing member 205A and the second stabilizing member 205B may expand to a diameter that is larger than the pericardial cavity 54 (e.g., the pericardial cavity 54 is forced to expand due to the force of the expanding stabilizing members). The force on the pericardial cavity walls causes the multi-lumen catheter 129 (and subsequently the blade 305) to be forced in the direction of the parietal layer 55. The first stabilizing member 205A and the second stabilizing member 205B are positioned radially about the assembly.

In one example, the first stabilizing member 205A and the second stabilizing member 205B are positioned radially about the assembly about 120 degrees apart. Additionally, the blade 305 may be positioned approximately 120 degrees from each of the first stabilizing member 205A and the second stabilizing member 205B, such that the first stabilizing member 205A and the second stabilizing member 205B assist the blade 305 cutting into the parietal layer 55 (e.g., the stabilizing member(s) 205A, 205B contact the pericardium walls, causing the multi-lumen catheter to receive a lateral force in the direction of the parietal layer 55). The first stabilizing member 205A and the second stabilizing member 205B can be flexible rods or strip, or inflatable structures, such as balloons that can be inflated with air or liquid (saline).

As shown in FIG. 8, the first stabilizing member 205A and the second stabilizing member 205B may be positioned radially around the multi-lumen catheter device 129. For example, the first stabilizing member 205A and the second stabilizing member 205B are spaced approximately 180 degrees from one another. Each of the first stabilizing member 205A and the second stabilizing member 205B have a wire or balloon structure that extends to contact walls of the pericardial cavity. In an example embodiment, the first stabilizing member 205A extends in the direction of the parietal layer 55. Additionally, the second stabilizing member 205B extends in the direction of the visceral layer 53. As such, the first stabilizing member 205A comes into contact with the parietal layer 55 and the second stabilizing member 205B comes into contact with the visceral layer 53.

The first stabilizing member 205A may have a cutting surface 805 on the balloon. The cutting surface 805 may be a blade or a surface of an electrode structured to receive sufficient current or radio frequency energy (RF) to ablate, burn, vaporize, or separate tissue. The cutting surface 805 may cause an incision into the parietal layer 55 as discussed above in reference to the blade 305 discussed herein. For example, the multi-lumen catheter 129 may be moved along the pericardial cavity 54, causing the cutting surface 805 to cut the incision along the parietal layer 55. Upon making the incision into the parietal layer 55, the first stabilizing member 205A and the second stabilizing member 205B may be moved back to the stabilizing retracted position to be removed from the human.

In one example, a puncture to deliver a guidewire into the pericardial space is performed through heart tissue. When a transvascular approach through the RAA, IVC, or SVC is employed, a closure device may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

In one example, to provide orientational stability of the cutting surface to that of the parietal layer, an OTW introduction is employed for any of the previously disclosed devices, for example, whether through a dedicated lumen in multi-lumen catheter cross-section or ‘Rapid Exchange’ style catheter, or off-center attached cannula, or deflect-resistant catheter. In one example, the delivering catheter comprises radiopaque material randomly distributed or arranged in a pattern for visualization using conventional visualization techniques during use.

Current ECHO/fluoroscopy may not provide the required visualization for certain access applications of the presently disclosed transection devices, for example, gaining guidewire access to the pericardial cavity consistently and repeatedly may be desired. Thus, in one example, the multi-lumen catheter 129 coupled to the presently disclosed transection devices comprises direct visualization allowing the user to watch real-time the advancement of any of the presently disclosed transection devices traverse through various tissue layers until the desired location is reached. Changes in tissue layers that may not be visible under ECHO/fluoroscopy may be easily distinguishable under direct visualization such as tissue/bloodstream (vessel access), myocardium/pericardium (pericardial cavity access), myocardium/pericardium (outside pericardium), among other anatomical features.

Thus, in one example, the presently disclosed devices discussed above further comprise an optical channel in the multi-lumen catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen catheter to accommodate a lens coupled to a fiber optic cable, optionally with a light source, e.g., an LED.

In one example, a puncture to deliver a guidewire into the pericardial cavity 54 is performed through heart tissue in a transvascular approach. When a transvascular approach through the RAA, IVC, or SVC is employed, a closure device (e.g., occluder) may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

The following exemplary occlusion descriptions relate to a transvascular approach through the Inferior Vena Cava (IVC), Superior Vena Cava (SVC) or coronary sinus (CS) and out the right (RA) or right atrial appendage (RAA), using one of the aforementioned transection devices (e.g., incision device 140). In one example, an introducer/dilator 115 delivers a wire into the pericardial space through heart tissue. A closure or occlusion device is introduced for hemostasis during the procedure. The closure or occlusion device in one example includes outward or radially directed splines deployed in an expanded configuration. When the guide catheter is removed, the splines or radial members of the closure device contract inwardly towards the unstressed state of the transection device in order to close and seal the opening. The closure device is designed such that a pericardial cutting device can pass through and into the pericardial space.

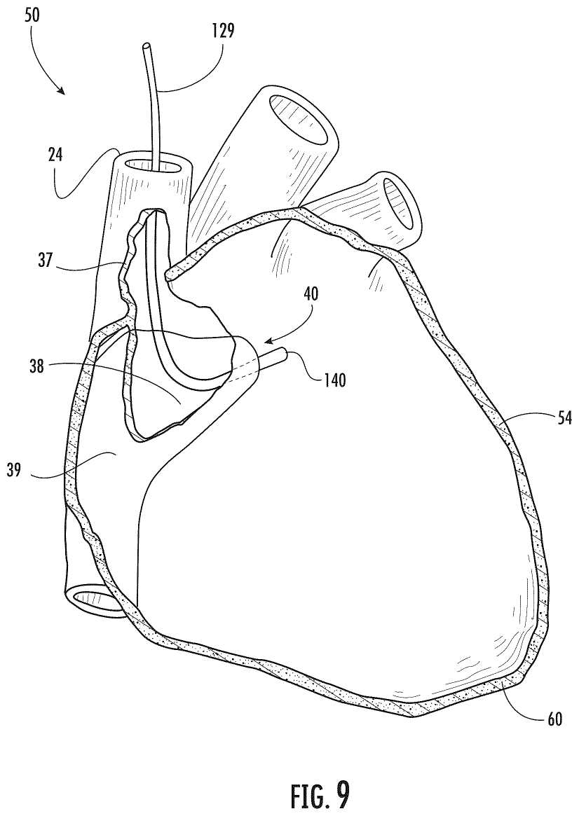

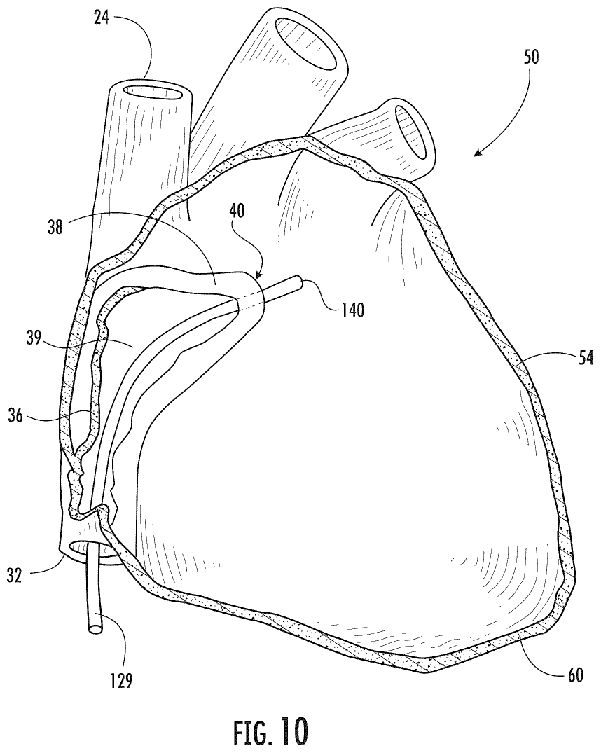

FIGS. 9 and 10 shows exemplary intravascular approaches for delivering the transection devices of the present disclosure to the pericardial cavity 54. Thus, FIG. 9 depicts heart 50 viewed in isolation from the body, with the pericardium 60 or pericardial sac encasing the cardiac muscle (i.e., epicardium, myocardium and endocardium). The small space which is present between the heart muscle and pericardium 60 represents the pericardial cavity 54.

The presently disclosed transection devices that can be presented to the pericardial cavity 54. In one example via the right atrial appendage 38 (RAA), which is a suitable site for entry into the pericardial cavity 54, is used. Right atrial appendage 38 lies tangential to and between pericardium 60 and the epicardium/epicardial adipose tissue 57. In one example, any of the presently disclosed devices is guided into right atrial appendage 38 via right atrium 39 so as to be positioned substantially in parallel with the wall of pericardium 60 such that when the wall of right atrial appendage 38 is pierced by any of the presently disclosed devices substantially without risk of damaging the epicardium or other heart tissue. Other access routes to the pericardial cavity can be used, for example, direct “puncture out” of SVC or IVC/coronary sinus (CS) and a “puncture into” the pericardium.

In some examples, right atrial appendage 38 is accessed via conventional vena cava routes. FIG. 9 illustrates entry of any of the presently disclosed devices into right atrium 39 via the superior vena cava 24 (SVC). A cut-away 37 shows passage of any of the presently disclosed devices through superior vena cava 24, right atrium 39, and right atrial appendage 38. A distal tip of catheter 129 is shown exiting right atrium 39 at apex 40.

FIG. 10 illustrates an alternative entry of any of the previously disclosed devices into right atrium 39 via the inferior vena cava 32 (IVC). A cut-away 36 shows passage of catheter 129 through inferior vena cava 32, right atrium 39, and right atrial appendage 38. A distal tip of catheter 129 is shown exiting right atrium 39 at apex 40.

Thus, by way of example, a method of reducing pericardial restraint of a subject in need thereof using any of the presently disclosed devices is provided by the following steps. Any of the presently disclosed devices is maneuvered through one of the vena cava 24, 32 to right atrium 39. Once inside right atrium 39, any of the presently disclosed devices is passed into the right atrial appendage 38. The wall of right atrial appendage 38 is pierced at apex 40, and the catheter is advanced into the pericardial cavity 54. Other transvascular-right heart routes to the pericardial cavity 54 are envisaged. Furthermore, left atrial appendage, coronary sinus, and right ventricle pathways are envisaged for transvascular access to the pericardial cavity 54.

Note that the wall of the right atrial appendage may be pierced with any of the presently disclosed devices itself, or with an instrument (e.g., guidewire) passed through a lumen of the any of the presently disclosed devices, e.g., over the wire. Further, any of the previously disclosed devices may be passed into the pericardial space through the opening in the wall of the atrial appendage, or an instrument passed through the lumen of any of the presently disclosed devices may be presented into the pericardial cavity 54. These details will depend on the procedure being performed and on the type of the previously disclosed device being employed.

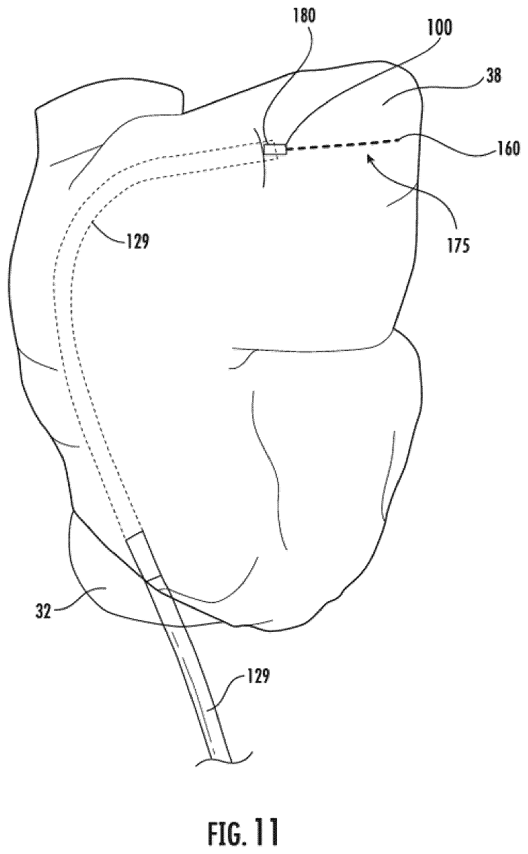

As shown in FIG. 11, any of the presently disclosed devices can be used to create a cut path of a length in a pericardium, e.g., in a parietal layer 58. Thus, a catheter 129, e.g., a steerable catheter can be employed, extending through the IVC, through the RA, and into the RAA, for example, and then into the pericardial cavity 54. The catheter 129 may have one or more steerable segments guiding any of the presently disclosed devices, with a radius of curvature of between about 1 inch and about 5 inches, with an arc length of between about 90° and about 180°. As exemplary shown in FIG. 11, any of the presently disclosed devices can be positioned in the pericardial cavity 54 and can begin a cut path 175 at a start point 160 and ends at endpoint 180 of a length. At least a portion of the parietal layer 55 of the pericardium, and the fibrous pericardium 56, and pericardial adipose tissue 59 are separated along cut path 175. The one or more incisions along the lengths, in a heart with a dysfunction treatable with the present method, cause the pericardium to separate radially about the cut line of the incision path 175, without the removal of pericardial tissue and with a reduction in pericardial restraint. One or more cut paths 175 can be made, and different cut paths, of various lengths can be used to reduce pericardial restraint. In one example, the cut path 175 and its length is pre-operatively determined. Other cut paths and lengths can be used.

In one example, the presently disclosed device further comprises at least one nerve detection device. In one example, the at least one nerve detection device is located on the flexible catheter 129. In one example, the at least one nerve detection device is located adjacent the incision device. In one example, the at least one nerve detection device is located on the dilator/introducer 115. In one example, the at least one nerve detection device is located on the cutting surface.

Any one of the presently disclosed devices can further comprises at least one nerve stimulation device. In one example, the at least one nerve stimulation device is located on the flexible catheter 129. In one example, the at least one nerve stimulation device is located adjacent the incision device. In one example, the at least one nerve stimulation device is located on introducer 115. In one example, the at least one nerve stimulation device is located on the cutting surface.

In one example, the presently disclosed devices discussed above further comprise an optical channel in the multi-lumen catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen catheter to accommodate a lens coupled to a fiber optic cable, optionally with a light source, e.g., an LED.

A kit, comprising any one of the presently disclosed medical devices, a sheath, a guidewire, and a puncturing tip is provided.

While certain embodiments of the present disclosure have been illustrated with reference to specific combinations of elements, various other combinations may also be provided without departing from the teachings of the present disclosure. Thus, the present disclosure should not be construed as being limited to the particular exemplary embodiments described herein and illustrated in the Figures, but may also encompass combinations of elements of the various illustrated embodiments and aspects thereof.

We claim:

1. A medical device comprising:
an incision device structured to be operably coupled to a distal end of a catheter, wherein the incision device comprises an incision channel defined along the incision device; and
a retractable cutting apparatus disposed within the incision channel, wherein the retractable cutting apparatus comprises:
a gear-actuated blade structured to move between a retracted position and a deployed position within the incision channel.

2. The medical device of claim 1, wherein in an instance in which the gear-actuated blade is in the retracted position, the gear-actuated blade is disposed within the incision channel, and wherein in an instance in which the gear-actuated blade is in the deployed position, the gear-actuated blade at least partially protrudes from the incision channel, and wherein said medical device further comprises:
a torsion gear mechanism attached to the gear-actuated blade, wherein the torsion gear is structured to move the gear-actuated blade between the retracted position and the deployed position; and
a rotatable gear actuator that is structured to move the torsion gear mechanism.

3. The medical device of claim 1, wherein the retractable cutting apparatus comprises a force transferring gear mechanism connected between the torsion gear mechanism and the rotatable gear actuator.

4. The medical device of claim 3, wherein the force transferring gear mechanism is a worm gear.

5. The medical device of claim 3, wherein the retractable cutting apparatus comprises an intermediate actuation member, wherein the intermediate actuation member is disposed between the force transferring gear mechanism and the torsion gear mechanism.

6. The medical device of claim 2, wherein the torsion gear mechanism is a rack and pinion gear.

7. The medical device of claim 6, wherein a rack portion of the rack and pinion gear is attached to the gear-actuated blade.

8. The medical device of claim 7, wherein the gear-actuated blade is structured to move along a latitudinal axis upon actuation of the torsion gear mechanism.

9. The medical device of claim 8, wherein a pinion portion of the rack and pinion gear is a pinion gear is attached to the rotatable gear actuator, wherein rotation of the rotatable gear actuator causes the rack portion to move along the pinion portion of the rack and pinion gear.

10. The medical device of claim 8, wherein a pinion portion of the rack and pinion gear is attached to the intermediate actuation member.

11. The medical device of claim 5, wherein the intermediate actuation member defines a proximal end and a distal end, wherein at least a portion of the force transferring gear mechanism is attached to the proximal end of the intermediate actuation member and at least a portion of the torsion gear mechanism is attached to the distal end of the intermediate actuation member.

12. The medical device of claim 1, further comprising one or more stabilizing members situated along the incision device, wherein the one or more stabilizing members is structured to maintain the incision device in place during an incision by the gear-actuated blade.

13. The medical device of claim 1, wherein the gear-actuated blade comprises at least one of a cutting surface or an electrode.

14. The medical device of claim 1, wherein the gear-actuated blade is reversibly adjustable laterally relative to the longitudinal axis of the multi-lumen catheter between a range of angles.

15. The medical device of claim 1, further comprising a controller engaged to the incision device, wherein the controller is structured to provide at least one of movement of the rotatable gear actuator, movement of a stabilizing member between a retracted position and a deployed position, movement of the incision device, movement of the catheter, or energy to the incision device.

16. A medical device comprising:
an incision device structured to be operably coupled to a distal end of a catheter, wherein the incision device comprises an incision channel defined along the incision device;
a retractable cutting apparatus disposed within the incision channel, wherein the retractable cutting apparatus comprises:
a gear-actuated blade structured to move between a retracted position and a deployed position within the incision channel;
a torsion gear mechanism attached to the gear-actuated blade, wherein the torsion gear is structured to move the gear-actuated blade between the retracted position and the deployed position;
a rotatable gear actuator that is structured to move the torsion gear mechanism;
a force transferring gear mechanism connected between the torsion gear mechanism and the rotatable gear actuator; and
an intermediate actuation member, wherein the intermediate actuation member is disposed between the force transferring gear mechanism and the torsion gear mechanism; and
one or more stabilizing members situated along the incision device, wherein the one or more stabilizing members is structured to maintain the incision device in place during an incision by the gear-actuated blade.

17. The medical device of claim 16, wherein the torsion gear mechanism is a rack and pinion gear.

18. The medical device of claim 17, wherein a rack portion of the rack and pinion gear is attached to the gear-actuated blade.

19. The medical device of claim 18, wherein a pinion portion of the rack and pinion gear is a pinion gear is attached to the rotatable gear actuator, wherein rotation of the rotatable gear actuator causes the rack portion to move along the pinion portion of the rack and pinion gear.

20. The medical device of claim 18, wherein a pinion portion of the rack and pinion gear is attached to the intermediate actuation member.
